SEQUENCE LISTING (20 sequences)

SEQ ID NO: 1 - protein, 89 aa, Artificial sequence
  Synthetic
GAIEVKDVTDTTALITWAKPWVDPPPLWGCELTYGIKDVPGDRTTIDLQQKHTAYSIGNLKPDTEYEVSLICFDPYGMRSKPAKETFTT

SEQ ID NO: 2 - protein, 89 aa, Artificial sequence
  Synthetic
GAIEVKDVTDTTALITWAKPWVDPPPLWGIELTYGIKDVPGDRTTIDLQQKHTAYSIGNLKPDTEYEVSLISFDPYGMRSKPAKETFTT

SEQ ID NO: 3 - protein, 15 aa, Artificial sequence
  Synthetic
GGGGSGGGGSGGGGS

SEQ ID NO: 4 - protein, 19 aa, Artificial sequence
  Synthetic
PQPQPKPQPKPEPEPQPQG

SEQ ID NO: 5 - protein, 60 aa, Artificial sequence
  Synthetic
GVDNKFNKEMWAAWEEIRNLPNLNGWQMTAFIASLVDDPSQSANLLAEAKKLNDAQAPKG

SEQ ID NO: 6 - protein, 8 aa, Artificial sequence
  Synthetic
HHHHHHHH

SEQ ID NO: 7 - protein, 600 aa, Artificial sequence
  Synthetic
VGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPG

SEQ ID NO: 8 - protein, 600 aa, Artificial sequence
  Synthetic
VPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAG

SEQ ID NO: 9 - protein, 300 aa, Artificial sequence
  Synthetic
VGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPG

SEQ ID NO: 10 - protein, 1223 aa, Artificial sequence
  Synthetic
VGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGS

SEQ ID NO: 11 - protein, 1223 aa, Artificial sequence
  Synthetic
VPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGSGAIEVKDVTDTTALITWAKPWVDPPPLWGCELTYGIKDVPGDRTTIDLQQKHTAYSIGNLKPDTEYEVSLICFDPYGMRSKPAKETFTTGGGGSGGGGSGGGGS

SEQ ID NO: 12 - DNA, 5298 bp, Artificial sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggaggagtacatatgggctgatgataatgatcttcaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 13 - DNA, 353 bp, Artificial sequence
  Synthetic
taagaaggaggagtacatatgggcgctatcgaagttaaagacgttaccgacaccaccgctctgatcacctgggctaaaccgtgggttgacccgccgccgctgtggggttgcgaactgacctacggtatcaaagacgttccgggtgaccgtaccaccatcgacctgcagcagaaacacaccgcttactctatcggtaacctgaaaccggacaccgaatacgaagtttctctgatctgcttcgacccgtacggtatgcgttctaaaccggctaaagaaaccttcaccaccggtggtggtggttctggtggtggtggttctggtggtggtggttctggcatatgtactcctcctta

SEQ ID NO: 14 - DNA, 262 bp, Artificial sequence
  Synthetic
agaaataattttgtttaactttaagaaggaggagtacatatgggcgttgataacaaattcaataaagaaatgtgggcagcctgggaagaaattcgtaacctgccgaacctgaatggttggcaaatgaccgccttcattgcgagcctggtggatgatccgagccaaagcgctaatctgctggcggaagcgaaaaaactgaacgacgcccaagccccgaaaggctgataataatgatcttcaggatccgaattcgagctccgtc

SEQ ID NO: 15 - DNA, 26 bp, Artificial sequence
  Synthetic
taagaaggaggagtacatatgggcgc

SEQ ID NO: 16 - DNA, 28 bp, Artificial sequence
  Synthetic
taaggaggagtacatatgccagaaccac

SEQ ID NO: 17 - protein, 4 aa, Artificial sequence
  Synthetic
RGDS

SEQ ID NO: 18 - protein, 17 aa, Artificial sequence
  Synthetic
  Xaa (2)..(16): any amino acid wherein none, any one or all of amino acids at positions 2-16 can either be present or absent
PXXXXXXXXXXXXXXXG

SEQ ID NO: 19 - protein, 5 aa, Artificial sequence
  Synthetic
  Xaa (4)..(4): any amino acid except proline
VPGXG

SEQ ID NO: 20 - protein, 47 aa, Artificial sequence
  Synthetic
  Xaa (1)..(15): any one or all of amino acid at positions 1-15 may either be present or absent
  Xaa (17)..(31): any one or all of amino acid at positions 17-31 may either be present or absent
  Xaa (33)..(47): any one or all of amino acid at positions 33-47 may either be present or absent
XXXXXXXXXXXXXXXPXXXXXXXXXXXXXXXGXXXXXXXXXXXXXXX

(19) United States
(12) Patent Application Publication
(10) Pub. No.: US 2018/0258157 A1
(43) Pub. Date: Sep. 13, 2018

(54) TARGETED THERAPEUTIC AGENTS COMPRISING MULTIVALENT PROTEIN-BIOPOLYMER FUSIONS

(71) Applicant: Duke University, Durham, NC (US)

(72) Inventors: Ashutosh Chilkoti, Durham, NC (US); Mandana Manzari, Durham, NC (US); Mareva Fevre, San Jose, CA (US)

(21) Appl. No.: 15/561,799

(22) PCT Filed: Mar. 25, 2016
(86) PCT No.: PCT/US2016/024202

§ 371 Date: Sep. 26, 2017

Related U.S. Application Data
(60) Provisional application No. 62/138,847, filed on Mar. 26, 2015.

(51) Int. Cl. C07K 14/78; A61P 35/00; A61K 9/00

(52) U.S. Cl. CPC C07K 14/78; A61P 35/00; A61K 38/00; C07K 2319/01; A61K 9/0019

(57) ABSTRACT

Provided herein are fusion proteins including at least one binding polypeptide and at least one unstructured polypeptide. The fusion protein may further include at least one linker. Further provided are methods for determining the presence of a target in a sample, methods of treating a disease, methods of diagnosing a disease in a subject, and methods of determining the effectiveness of a treatment for a disease in a subject. The methods may include administering to the subject an effective amount of the fusion protein.

### CROSS-REFERENCE TO RELATED APPLICATIONS

This application claims priority to U.S. Provisional Patent Application No. 62/138,847, filed Mar. 26, 2015, which is incorporated herein by reference in its entirety.

### STATEMENT REGARDING FEDERALLY SPONSORED RESEARCH

This invention was made with government support under grant RO1 EB007205, 2032358, and 2032363 awarded by the National Institutes of Health. The government has certain rights in the invention.

### FIELD

The disclosure relates to antibody mimetics and, more particularly, to fusions of unstructured polypeptides and multivalent proteins that specifically bind a target. The multivalent proteins can bind a target such as a cell surface receptor, for example, and thereby affect cellular physiology. The unstructured polypeptide component can render the fusion protein environmentally responsive, and thereby expand the scope of drug delivery options.

### INTRODUCTION

Proteins can be powerful therapeutic agents when engineered for affinity, specificity, and selectivity for a clinical target. Their complexity, versatility, tolerability, and diversity often make them superior alternatives to small molecule drugs, and the long half-life, specificity, and selectivity of some proteins make them attractive for some therapies. Biotechnological advances have enabled the engineering of proteins with specific properties and the manipulation of existing proteins for maximum therapeutic potential. Although protein engineering allows for the development of potent therapeutics targeted toward a protein or receptor of interest, the body has many mechanisms with which to clear such protein therapies. Thus, delivery is a critical issue for effectively translating a protein therapeutic to the clinic. There is a need for reliable and broadly applicable protein delivery solutions.

### SUMMARY

In an aspect, provided herein are fusion proteins. The fusion protein may include at least one binding polypeptide and at least one unstructured polypeptide. In some embodiments, the fusion protein comprises a plurality of unstructured polypeptides. In some embodiments, the fusion protein comprises a plurality of binding polypeptides. In some embodiments, the fusion protein further includes a linker positioned between at least two adjacent binding polypeptides. In some embodiments, the fusion protein further includes a linker positioned between at least two adjacent unstructured polypeptides. In some embodiments, the linker comprises at least one glycine and at least one serine. In some embodiments, the linker comprises an amino acid sequence consisting of SEQ ID NO: 3 ((Gly₄Ser)₃). In some embodiments, the linker comprises an amino acid sequence consisting of SEQ ID NO: 4. In some embodiments, the plurality of binding polypeptides forms an oligomer. In some embodiments, the binding polypeptide binds a target. In some embodiments, the fusion protein binds more than one target. In some embodiments, the at least one binding polypeptide comprises a Fibronectin type III (FnIII) domain. In some embodiments, the FnIII domain binds TNF-related apoptosis-inducing ligand receptor 2 (TRAILR-2). In some embodiments, the at least one binding polypeptide comprises at least one amino acid sequence of consisting of SEQ ID NO: 17 (RGDS). In some embodiments, the at least one binding polypeptide comprises a plurality of amino acid sequences consisting of SEQ ID NO: 17 (RGDS). In some embodiments, the at least one unstructured polypeptide comprises at least one PG motif comprising an amino acid sequence selected from PG, P(X)ₙG (SEQ ID NO: 18), and (U)ₘP(X)ₙG(Z)ₚ (SEQ ID NO: 20), or a combination thereof, wherein m, n, and p are independently an integer from 1 to 15, and wherein U, X, and Z are independently any amino acid. In some embodiments, the at least one unstructured polypeptide includes a thermally responsive polypeptide. In some embodiments, the thermally responsive polypeptide comprises an elastin-like polypeptide (ELP). In some embodiments, the at least one unstructured polypeptide includes an amino acid sequence consisting of (VPGXG)ₙ (SEQ ID NO: 19), wherein X is any amino acid except proline and n is an integer greater than or equal to 1. In some embodiments, n is 60, 120, or 180. In some embodiments, X is valine. In some embodiments, the fusion protein further includes at least one linker positioned between the at least one binding polypeptide and the at least one unstructured polypeptide. In some embodiments, the fusion protein includes a plurality of linkers between the at least one binding polypeptide and the at least one unstructured polypeptide. In some embodiments, the at least one binding polypeptide is positioned N-terminal to the at least one unstructured polypeptide. In some embodiments, the at least one binding polypeptide is positioned C-terminal to the at least one unstructured polypeptide. In some embodiments, the at least one unstructured polypeptide has a LCST between about 0° C. and about 100° C. In some embodiments, the at least one unstructured polypeptide has a UCST between about 0° C. and about 100° C.

In another aspect, provided herein are methods for treating a disease in a subject in need thereof. The method may include administering to the subject an effective amount of the fusion protein as described herein. In some embodiments, the fusion protein is administered in a controlled release formulation. In some embodiments, the fusion protein forms a depot upon administration to the subject. In some embodiments, the fusion protein is administered intravenously, intraarterially, or intraperitoneally to the subject. In some embodiments, the disease includes cancer. In some embodiments, the fusion protein is administered intratumorally. In some embodiments, the cancer is colorectal adenocarcinoma. In some embodiments, the at least one binding polypeptide includes an FnIII domain or a plurality of FnIII domains, and the disease is a disease associated with TRAILR-2. In some embodiments, the disease is a disease associated with a target of the at least one binding polypeptide.

In another aspect, provided herein are multivalent fusion proteins. The multivalent fusion protein may include at least one Fibronectin type III (FnIII) domain and at least one elastin-like polypeptide (ELP). In some embodiments, the FnIII domain binds TNF-related apoptosis-inducing ligand receptor 2 (TRAILR-2). In some embodiments, the at least one ELP includes an amino acid sequence consisting of (VPGXG)ₙ (SEQ ID NO: 19), wherein X is any amino acid except proline and n is an integer greater than or equal to 1. In some embodiments, n is 60, 120, or 180. In some embodiments, X is valine. In some embodiments, the at least one FnIII domain includes an amino acid sequence consisting of SEQ ID NO: 1. In some embodiments, the multivalent fusion protein includes a plurality of FnIII domains. In some embodiments, the multivalent fusion protein includes 2, 4, or 6 FnIII domains. In some embodiments, the multivalent fusion protein further includes a linker positioned between at least two adjacent FnIII domains. In some embodiments, the linker includes at least one glycine and at least one serine. In some embodiments, the linker includes an amino acid sequence consisting of SEQ ID NO: 3 ((Gly₄Ser)₃). In some embodiments, the linker includes an amino acid sequence consisting of SEQ ID NO: 4.

In another aspect, provided herein are methods for treating a disease associated with TNF-related apoptosis-inducing ligand receptor 2 (TRAILR-2) in a subject in need thereof. The methods may include administering to the subject an effective amount of the multivalent fusion protein as detailed herein. In some embodiments, the disease includes cancer. In some embodiments, the cancer includes colorectal adenocarcinoma. In some embodiments, the multivalent fusion protein is administered intravenously, intraarterially, or intraperitoneally to the subject. In some embodiments, the multivalent fusion protein is administered intratumorally. In some embodiments, the multivalent fusion protein forms a depot upon administration to the subject. In some embodiments, the multivalent fusion protein is administered in a controlled release formulation.

In another aspect, provided herein are methods of diagnosing a disease in a subject. The method may include contacting a sample from the subject with a fusion protein as detailed herein, and detecting binding of the fusion protein to a target to determine presence of the target in the sample, wherein the presence of the target in the sample indicates the disease in the subject. In some embodiments, the disease is selected from cancer, metabolic disease, autoimmune disease, cardiovascular disease, and orthopedic disorder.

In another aspect, provided herein are methods of determining the presence of a target in a sample. The method may include contacting the sample with a fusion protein as detailed herein under conditions to allow a complex to form between the fusion protein and the target in the sample, and detecting the presence of the complex, wherein presence of the complex is indicative of the target in the sample. In some embodiments, the sample is obtained from a subject and the method further includes diagnosing a disease, prognosticating, or assessing the efficacy of a treatment of the subject. In some embodiments, the method further includes assessing the efficacy of a treatment of the subject, and the method further includes modifying the treatment of the subject as needed to improve efficacy.

In another aspect, provided herein are methods of determining the effectiveness of a treatment for a disease in a subject in need thereof. The method may include contacting a sample from the subject with a fusion protein as described herein under conditions to allow a complex to form between the fusion protein and a target in the sample, determining the level of the complex in the sample, wherein the level of the complex is indicative of the level of the target in the sample, and comparing the level of the target in the sample to a control level of the target, wherein if the level of the target is different from the control level, then the treatment is determined to be effective or ineffective in treating the disease. In some embodiments, the method further includes modifying the treatment or administering a different treatment to the subject when the treatment is determined to be ineffective in treating the disease.

In another aspect, provided herein are methods of diagnosing a disease in a subject. The method may include contacting a sample from the subject with a fusion protein as described herein, determining the level of a target in the sample, and comparing the level of the target in the sample to a control level of the target, wherein a level of the target different from the control level indicates disease in the subject.

In some embodiments, the control level corresponds to the level in the subject at a time point before or during the period when the subject has begun treatment, and the sample is taken from the subject at a later time point. In some embodiments, the sample is taken from the subject at a time point during the period when the subject is undergoing treatment, and the control level corresponds to a disease-free level or to the level at a time point before the period when the subject has begun treatment.

In some embodiments, the fusion protein is labeled with a reporter. In some embodiments, the disease is selected from cancer, metabolic disease, autoimmune disease, cardiovascular disease, and orthopedic disorder.

### BRIEF DESCRIPTION OF THE DRAWINGS

FIG. 1 is a schematic illustrating the architecture of the protein biopolymer fusions. The multivalent protein drugs can act as agonists to amplify receptor signaling or as antagonists to inhibit ligand binding and prevent receptor signaling.

FIG. 2 is a schematic of the multivalent TRAILR-2 agonist-ELP constructs. The Tn3-ELP fusions were constructed to express ELPs at the N-terminus (shown) or C-terminus (not shown). Each Tn3 unit had a molecular weight of approximately 10 kDa, and the molecular weight of the ELPs varied.

FIG. 3 shows SDS-PAGE analysis of the ELP-TRAILR-2 agonist fusion protein at various steps in the purification process. Lanes: 1: Cell lysate; 2: hot spin 1 supernatant; 3: hot spin 1 pellet; 4: cold spin 2 supernatant; 5: hot spin 2 pellet; 6: purified product (cold spin 3 supernatant); 7: purified product (cold spin 3 supernatant). Samples in lanes 1-6 contained reducing agent; lane 7 did not.

FIG. 4 is a graph showing that tetravalent TRAILR-2-ELPa-(Tn3)₄ fusions inhibited cell viability of Colo205 human colorectal adenocarcinoma cells and outperformed TRAIL. Hexavalent TRAILR-2-ELPa-(Tn3)₆ fusions exhibited potent activation of apoptosis as well. Presence of ELP did not affect the potency of the drug.

FIG. 5 is a graph showing the transition temperatures. The transition temperature of the 6 repeat agonist ELP fusion was 29.2° C., and the transition temperature of the 4 repeat agonist ELP fusion was 27.9° C. This range was appropriate for s.c./intratumoral injections in mouse Colo205 xenograft models.

FIG. 6 is a graph showing the changes in tumor volume in Colo205 colorectal cancer xenograft models in response to multivalent TRAILR-2 specific ELP fusions. Tumors in mice treated with depot-forming ELPa-(Tn3)₆ fusions underwent partial regression and delayed growth.

### DETAILED DESCRIPTION

Provided herein are compositions and methods for delivering protein therapeutics to a subject. The compositions and methods include a fusion protein. The fusion protein may include a binding polypeptide fused to an unstructured polypeptide. In some embodiments, the unstructured polypeptide may include a thermally responsive protein polymer, which may facilitate slow release from a gel-like depot. The use of protein drugs, particularly antibodies, has led to many successful treatments. The long half-life, specificity, and selectivity of engineered antibodies make them excellent for some therapies. The limitations of architecture, valency, potency, aggregation, and manufacturing cost of antibodies can be major hindrances in translation to the clinic. The compositions and methods detailed herein may overcome these limitations and facilitate the use of protein therapeutics for clinical use. The fusion proteins may allow for the treatment of disease by effectively delivering binding polypeptides so they may associate with their target to treat the disease. The fusion proteins may also be used to detect a target, detect or diagnose disease, and/or determine the efficacy of a treatment.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Affinity” refers to the binding strength of a binding polypeptide to its target (i.e., binding partner).

“Agonist” refers to an entity that binds to a receptor and activates the receptor to produce a biological response. An “antagonist” blocks or inhibits the action or signaling of the agonist. An “inverse agonist” causes an action opposite to that of the agonist. The activities of agonists, antagonists, and inverse agonists may be determined in vitro, in situ, in vivo, or a combination thereof.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “biomarker” refers to a naturally occurring biological molecule present in a subject at varying concentrations that is useful in identifying and/or classifying a disease or a condition. The biomarker can include genes, proteins, polynucleotides, nucleic acids, ribonucleic acids, polypeptides, or other biological molecules used as an indicator or marker for disease. In some embodiments, the biomarker comprises a disease marker. For example, the biomarker can be a gene that is upregulated or downregulated in a subject that has a disease. As another example, the biomarker can be a polypeptide whose level is increased or decreased in a subject that has a disease or risk of developing a disease. In some embodiments, the biomarker comprises a small molecule. In some embodiments, the biomarker comprises a polypeptide.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice.

The term “expression vector” indicates a plasmid, a virus or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Polymer” as used herein is intended to encompass a homopolymer, heteropolymer, block polymer, co-polymer, ter-polymer, etc., and blends, combinations and mixtures thereof. Examples of polymers include, but are not limited to, functionalized polymers, such as a polymer comprising 5-vinyltetrazole monomer units and having a molecular weight distribution less than 2.0. The polymer may be or contain one or more of a star block copolymer, a linear polymer, a branched polymer, a hyperbranched polymer, a dendritic polymer, a comb polymer, a graft polymer, a brush polymer, a bottle-brush copolymer and a crosslinked structure, such as a block copolymer comprising a block of 5-vinyltetrazole monomer units. Polymers include, without limitation, polyesters, poly(meth)acrylamides, poly(meth)acrylates, polyethers, polystyrenes, polynorbornenes and monomers that have unsaturated bonds. For example, amphiphilic comb polymers are described in U.S. Patent Application Publication No. 2007/0087114 and in U.S. Pat. No. 6,207,749 to Mayes et al., the disclosure of each of which is herein incorporated by reference in its entirety. The amphiphilic comb-type polymers may be present in the form of copolymers, containing a backbone formed of a hydrophobic, water-insoluble polymer and side chains formed of short, hydrophilic non-cell binding polymers. Examples of other polymers include, but are not limited to, polyalkylenes such as polyethylene and polypropylene; polychloroprene; polyvinyl ethers; such as poly(vinyl acetate); polyvinyl halides such as poly(vinyl chloride); polysiloxanes; polystyrenes; polyurethanes; polyacrylates; such as poly(methyl (meth)acrylate), poly(ethyl (meth)acrylate), poly(n-butyl(meth)acrylate), poly(isobutyl (meth)acrylate), poly(tert-butyl (meth)acrylate), poly(hexyl(meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), and poly(octadecyl acrylate); polyacrylamides such as poly(acrylamide), poly(methacrylamide), poly(ethyl acrylamide), poly(ethyl methacrylamide), poly(N-isopropyl acrylamide), poly(n, iso, and tert-butyl acrylamide); and copolymers and mixtures thereof. These polymers may include useful derivatives, including polymers having substitutions, additions of chemical groups, for example, alkyl groups, alkylene groups, hydroxylations, oxidations, and other modifications routinely made by those skilled in the art. The polymers may include zwitterionic polymers such as, for example, polyphosphorycholine, polycarboxybetaine, and polysulfobetaine. The polymers may have side chains of betaine, carboxybetaine, sulfobetaine, oligoethylene glycol (OEG), sarcosine or polyethyleneglycol (PEG). For example, poly(oligoethyleneglycol methacrylate) (poly(OEGMA)) may be used. Poly(OEGMA) may be hydrophilic, water-soluble, non-fouling, non-toxic and non-immunogenic due to the OEG side chains.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units.

“Reporter,” “reporter group,” “label,” and “detectable label” are used interchangeably herein. The reporter is capable of generating a detectable signal. The label can produce a signal that is detectable by visual or instrumental means. A variety of reporter groups can be used, differing in the physical nature of signal transduction (e.g., fluorescence, electrochemical, nuclear magnetic resonance (NMR), and electron paramagnetic resonance (EPR)) and in the chemical nature of the reporter group. Various reporters include signal-producing substances, such as chromagens, fluorescent compounds, chemiluminescent compounds, radioactive compounds, and the like. In some embodiments, the reporter comprises a radiolabel. Reporters may include moieties that produce light, e.g., acridinium compounds, and moieties that produce fluorescence, e.g., fluorescein. In some embodiments, the signal from the reporter is a fluorescent signal. The reporter may comprise a fluorophore. Examples of fluorophores include, but are not limited to, acrylodan (6-acryloyl-2-dimethylaminonaphthalene), badan (6-bromo-acetyl-2-dimethylamino-naphthalene), rhodamine, naphthalene, danzyl aziridine, 4-[N-[(2-iodoacetoxy)ethyl]-N-methylamino]-7-nitrobenz-2-oxa-1,3-diazole ester (IANBDE), 4-[N-[(2-iodoacetoxy)ethyl]-N-methylamino-7-nitrobenz-2-oxa-1,3-diazole (IANBDA), fluorescein, dipyrrometheneboron difluoride (BODIPY), 4-nitrobenzo[c][1,2,5]oxadiazole (NBD), Alexa fluorescent dyes, and derivatives thereof. Fluorescein derivatives may include, for example, 5-fluorescein, 6-carboxyfluorescein, 3′6-carboxyfluorescein, 5(6)-carboxyfluorescein, 6-hexachlorofluorescein, 6-tetrachlorofluorescein, fluorescein, and isothiocyanate.

“Sample” or “test sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “sensitivity” as used herein refers to the number of true positives divided by the number of true positives plus the number of false negatives, where sensitivity (“sens”) may be within the range of 0<sens<1. Ideally, method embodiments herein have the number of false negatives equaling zero or close to equaling zero, so that no subject is wrongly identified as not having a disease when they indeed have the disease. Conversely, an assessment often is made of the ability of a prediction algorithm to classify negatives correctly, a complementary measurement to sensitivity.

The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that a polypeptide binds to a target when it binds to that target more readily than it would bind to a random, unrelated target.

“Subject” as used herein can mean a mammal that wants or is in need of the herein described fusion proteins. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Transition” or “phase transition” refers to the aggregation of the thermally responsive polypeptides. Phase transition occurs sharply and reversibly at a specific temperature called the lower critical solution temperature (LCST) or the inverse transition temperature Tₜ. Below the transition temperature, the thermally responsive polypeptide (or a polypeptide comprising a thermally responsive polypeptide) is highly soluble. Upon heating past the transition temperature, the thermally responsive polypeptides hydrophobically collapse and aggregate, forming a separate, gel-like phase. “Inverse transition cycling” refers to a protein purification method for thermally responsive polypeptides (or a polypeptide comprising a thermally responsive polypeptide). The protein purification method may involve the use of thermally responsive polypeptide's reversible phase transition behavior to cycle the solution through soluble and insoluble phases, thereby removing contaminants.

“Treatment” or “treating,” when referring to protection of a subject from a disease, means preventing, suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Valency” as used herein refers to the potential binding units or binding sites. The term “multivalent” refers to multiple potential binding units. The terms “multimeric” and “multivalent” are used interchangeably herein.

“Variant” used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### 2. Fusion Protein

The fusion protein includes at least one binding polypeptide and at least one unstructured polypeptide. The fusion protein may further include at least one linker.

In some embodiments, the fusion protein includes more than one binding polypeptide. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 binding polypeptides. The fusion protein may include less than 30, less than 25, or less than 20 binding polypeptides. The fusion protein may include between 1 and 30, between 1 and 20, or between 1 and 10 binding polypeptides. In such embodiments, the binding polypeptides may be the same or different from one another. In some embodiments, the fusion protein includes more than one binding polypeptide positioned in tandem to one another. In some embodiments, the fusion protein includes 2 to 6 binding polypeptides. In some embodiments, the fusion protein includes two binding polypeptides. In some embodiments, the fusion protein includes three binding polypeptides. In some embodiments, the fusion protein includes four binding polypeptides. In some embodiments, the fusion protein includes five binding polypeptides. In some embodiments, the fusion protein includes six binding polypeptides.

In some embodiments, the fusion protein includes more than one unstructured polypeptide. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 unstructured polypeptides. The fusion protein may include less than 30, less than 25, or less than 20 unstructured polypeptides. The fusion protein may include between 1 and 30, between 1 and 20, or between 1 and 10 unstructured polypeptides. In such embodiments, the unstructured polypeptides may be the same or different from one another. In some embodiments, the fusion protein includes more than one unstructured polypeptide positioned in tandem to one another.

In some embodiments, the fusion protein may be arranged as a modular linear polypeptide. For example, the modular linear polypeptide may be arranged in one of the following structures: [binding polypeptide]ₘ-[linker]ₖ-[unstructured polypeptide]; [unstructured polypeptide]-[linker]ₖ-[binding polypeptide]ₘ; [binding polypeptide]ₘ-[linker]ₖ-[unstructured polypeptide]-[binding polypeptide]ₘ-[linker]ₖ-[unstructured polypeptide]; or [unstructured polypeptide]-[binding polypeptide]ₘ-[linker]ₖ-[unstructured polypeptide]-[binding polypeptide]ₘ, in which k and m are each independently an integer greater than or equal to 1. In some embodiments, m is an integer less than or equal to 20. In some embodiments, m is an integer equal to 1, 2, 3, 4, 5, 6, 7, 8, 9, 10, 11, 12, 13, 14, 15, 16, 17, 18, 19, or 20. In some embodiments, k is an integer less than or equal to 10. In some embodiments, k is an integer equal to 1, 2, 3, 4, 5, 6, 7, 8, 9, or 10. In some embodiments, the at least one binding polypeptide is positioned N-terminal to the at least one unstructured polypeptide. In some embodiments, the at least one binding polypeptide is positioned C-terminal to the at least one unstructured polypeptide.

The fusion protein may be expressed recombinantly in a host cell according to one of skill in the art. The fusion protein may be purified by any means known to one of skill in the art. For example, the fusion protein may be purified using chromatography, such as liquid chromatography, size exclusion chromatography, or affinity chromatography, or a combination thereof. In some embodiments, the fusion protein is purified without chromatography. In some embodiments, the fusion protein is purified using inverse transition cycling.

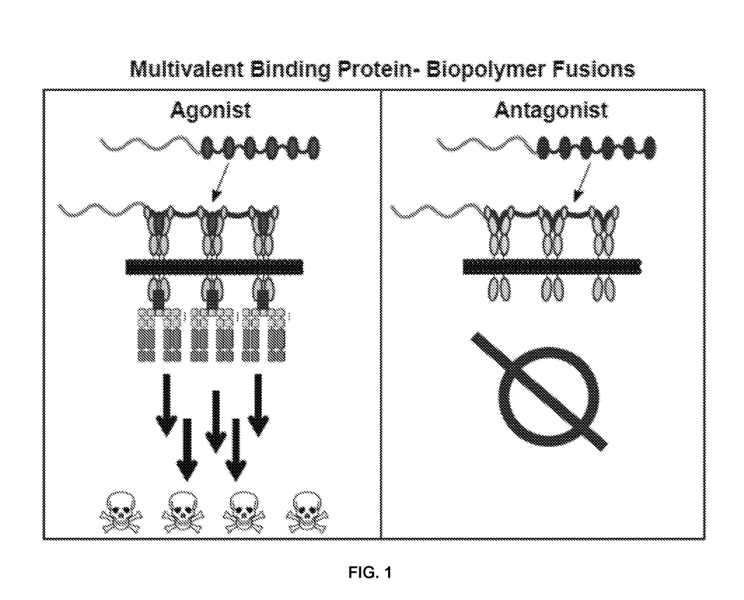

In some embodiments, the fusion protein comprises a plurality of binding polypeptides comprising Tn3 domains (SEQ ID NO: 1 or 2), linked to one another with flexible glycine serine linkers (SEQ ID NO: 3), and an unstructured polypeptide comprising elastin-like polypeptide (FIG. 1).

a. Binding Polypeptide

The binding polypeptide may comprise any polypeptide that is capable of binding at least one target. The binding polypeptide may bind at least one target. “Target” may be an entity capable of being bound by the binding polypeptide. Targets may include, for example, another polypeptide, a cell surface receptor, a carbohydrate, an antibody, a small molecule, or a combination thereof. The target may be a biomarker. The target may be activated through agonism or blocked through antagonism. The binding polypeptide may specifically bind the target. By binding target, the binding polypeptide may act as a targeting moiety, an agonist, an antagonist, or a combination thereof. In some embodiments, the binding polypeptide domain binds TRAILR-2. “TRAIL receptor 2” or “TRAILR-2” refers to the TNF-Related Apoptosis-Inducing Ligand (TRAIL) Receptor 2 protein. Upon binding TRAIL or other agonists, TRAILR-2 activates apoptosis, or programmed cell death, in tumor cells. In some embodiments, the binding polypeptide domain binds epidermal growth factor receptor (EGFR). Upon binding epidermal growth factor (EGF) and other growth factor ligands, EGFR activates signal transduction pathways that promote cell proliferation.

The binding polypeptide may be a monomer that binds to a target. The monomer may bind one or more targets. The binding polypeptide may form an oligomer. The binding polypeptide may form an oligomer with the same or different binding polypeptides. The oligomer may bind to a target. The oligomer may bind one or more targets. One or more monomers within an oligomer may bind one or more targets. In some embodiments, the fusion protein is multivalent. In some embodiments, the fusion protein binds multiple targets. In some embodiments, the activity of the binding polypeptide alone is the same as the activity of the binding protein when part of a fusion protein.

In some embodiments, the binding polypeptide comprises an amino acid sequence consisting of Arg-Gly-Asp-Ser (RGDS; SEQ ID NO: 17). In some embodiments, the binding polypeptide comprises a plurality of amino acid sequences consisting of SEQ ID NO: 17. The amino acid sequence of SEQ ID NO: 17 may be present anywhere within the binding polypeptide. In some embodiments, the amino acid sequence of SEQ ID NO: 17 may be repeated in tandem within the binding polypeptide.

In some embodiments, the binding polypeptide comprises one or more scaffold proteins. As used herein, “scaffold protein” refers to one or more polypeptide domains with relatively stable and defined three-dimensional structures. Scaffold proteins may further have the capacity for affinity engineering. In some embodiments, the scaffold protein has been engineered to bind a particular target. The scaffold proteins may be the same or different.

In some embodiments, the scaffold protein comprises a fibronectin domain. Fibronectin is a high-molecular weight glycoprotein of the extracellular matrix that binds to membrane-spanning receptor proteins called integrins. Fibronectin binds extracellular matrix components such as collagen, fibrin, and heparan sulfate proteoglycans. Human fibronectin exists as a protein dimer, comprising two nearly identical polypeptide chains linked by a pair of C-terminal disulfide bonds. Each human fibronectin subunit contains three domains: type I, II, and III. Fibronectin type III (FnIII) refers to the third of the three types of internal repeats in human fibronectin. This domain is often referred to as a scaffold protein because it contains three CDR-like (complementarity determining region) loops that can be engineered to bind a protein of interest using molecular biology techniques. In some embodiments, the fibronectin domain comprises Tn3. “Tn3” or “Tn3 scaffold” refers to an FnIII domain from human tenascin C. Tn3 may comprise an amino acid sequence consisting of SEQ ID NO: 1 or 2. In some embodiments, Tn3 binds TRAIL receptor 2. The binding polypeptide may comprise one or more scaffold proteins further selected from, for example, alphahelical Z domain of protein A, anti-EGFR binding protein, DARPINS, knottins, and scFvs.

b. Unstructured Polypeptide

The unstructured polypeptide may comprise any polypeptide that has minimal or no secondary structure as observed by CD, being soluble at a temperature below its lower critical solution temperature (LCST) and/or at a temperature above its upper critical solution temperature (UCST), and comprising a repeated amino acid sequence. LCST is the temperature below which the polypeptide is miscible. UCST is the temperature above which the polypeptide is miscible. In some embodiments, the unstructured polypeptide has only UCST behavior. In some embodiments, the unstructured polypeptide has only LCST behavior. In some embodiments, the unstructured polypeptide has both UCST and LCST behavior. The unstructured polypeptide may comprise a repeated sequence of amino acids. The unstructured polypeptide may have a LCST between about 0° C. and about 100° C., between about 10° C. and about 50° C., or between about 20° C. and about 42° C. The unstructured polypeptide may have a UCST between about 0° C. and about 100° C., between about 10° C. and about 50° C., or between about 20° C. and about 42° C. In some embodiments, the unstructured polypeptide has a transition temperature between room temperature (about 25° C.) and body temperature (about 37° C.). In some embodiments, a fusion protein comprising one or more thermally responsive polypeptides has a transition temperature between room temperature (about 25° C.) and body temperature (about 37° C.). In some embodiments, the unstructured polypeptide has no LCST or UCST behavior. The unstructured polypeptide may have its LCST or UCST below body temperature or above body temperature at the concentration at which the fusion protein is administered to a subject.

In some embodiments, the unstructured polypeptide comprises an amino acid sequence that is rich in proline and glycine. In some embodiments, the unstructured polypeptide comprises a PG motif. In some embodiments, the unstructured polypeptide comprises a plurality of or repeated PG motifs. A PG motif comprises an amino acid sequence selected from PG, P(X)ₙG (SEQ ID NO: 18), and (U)ₘP(X)ₙG(Z)ₚ (SEQ ID NO: 20), or a combination thereof, wherein m, n, and p are independently an integer from 1 to 15, and wherein U, X, and Z are independently any amino acid. P(X)ₙG may include PXG, PXXG, PXXXG, PXXXXG, PXXXXXG, PXXXXXXG, PXXXXXXXG, PXXXXXXXXG, PXXXXXXXXXG, PXXXXXXXXXXG, PXXXXXXXXXXXG, PXXXXXXXXXXXXG, PXXXXXXXXXXXXXG, PXXXXXXXXXXXXXXG, and/or PXXXXXXXXXXXXXXXG. The unstructured polypeptide may further include additional amino acids at the C-terminal and/or N-terminal end of the PG motif. These amino acids surrounding the PG motif may also be part of the overall repeated motif. The amino acids that surround the PG motif may balance the overall hydrophobicity and/or charge so as to control the LCST or UCST behavior of the unstructured polypeptide.

In some embodiments, the unstructured polypeptide comprises one or more thermally responsive polypeptides. Thermally responsive polypeptides may include, for example, elastin-like polypeptides (ELP). “ELP” refers to a polypeptide comprising the pentapeptide repeat sequence (VPGXG)ₙ, wherein X is any amino acid except proline and n is an integer greater than or equal to 1 (SEQ ID NO: 19). The unstructured polypeptide may comprise an amino acid sequence consisting of (VPGXG)ₙ. In some embodiments, X is not proline. In some embodiments, n is 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, n may be less than 500, less than 400, less than 300, less than 200, or less than 100. In some embodiments, n may be between 1 and 500, between 1 and 400, between 1 and 300, or between 1 and 200. In some embodiments, n is 60, 120, or 180. ELP may be expressed recombinantly.

Thermally responsive polypeptides, for example, ELP, may have a phase transition. The thermally responsive polypeptide may impart a phase transition characteristic to the unstructured polypeptide and/or fusion protein. “Phase transition” or “transition” may refer to the aggregation of the thermally responsive polypeptide, which occurs sharply and reversibly at a specific temperature called the lower critical solution temperature (LCST) or the inverse transition temperature (Tt). Below the transition temperature (LCST or Tt), the thermally responsive polypeptides (or polypeptides comprising a thermally responsive polypeptide) may be highly soluble. Upon heating above the transition temperature, thermally responsive polypeptides hydrophobically may collapse and aggregate, forming a separate, gel-like phase.

In other embodiments, the thermally responsive polypeptide comprises a resilin-like polypeptide (RLP). RLPs are derived from Rec1-resilin. Rec1-resilin is environmentally responsive and exhibits a dual phase transition behavior. The thermally responsive RLPs can have LCST and UCST (Li et. al, Macromol. Rapid Commun. 2015, 36, 90-95.) Additional examples of suitable thermally responsive polypeptides are described in U.S. Patent Application Publication Nos. US2012/0121709, filed May 17, 2012, and US2015/0112022, filed Apr. 23, 2015, each of which is incorporated herein by reference.

The thermally responsive polypeptides can phase transition at a variety of temperatures and concentrations. Thermally responsive polypeptides, for example, ELP, may not affect the binding or potency of the binding polypeptides. Thermally responsive polypeptides may allow the fusion protein to be tuned by a user to any number of desired transition temperatures, molecular weights, and formats.

Thermally responsive polypeptides may exhibit inverse phase transition behavior and thus, the fusion protein comprising the thermally responsive polypeptide may exhibit inverse phase transition behavior. Inverse phase transition behavior may be used to form drug depots within a tissue of a subject for controlled (slow) release of the fusion protein. Inverse phase transition behavior may also enable purification of the fusion protein using inverse transition cycling, thereby eliminating the need for chromatography.

c. Linker

In some embodiments, the fusion protein further includes at least one linker. In some embodiments, the fusion protein includes more than one linker. In such embodiments, the linkers may be the same or different from one another. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers. The fusion protein may include less than 500, less than 400, less than 300, or less than 200 linkers. The fusion protein may include between 1 and 1000, between 10 and 900, between 10 and 800, or between 5 and 500 linkers.

The linker may be positioned in between a binding polypeptide and an unstructured polypeptide, in between binding polypeptides, in between unstructured polypeptides, or a combination thereof. Multiple linkers may be positioned adjacent to one another. Multiple linkers may be positioned adjacent to one another and in between the binding polypeptide and the unstructured polypeptide.

The linker may be a polypeptide of any amino acid sequence and length. The linker may act as a spacer peptide. The linker may occur between polypeptide domains. The linker may sufficiently separate the binding domains of the binding polypeptide while preserving the activity of the binding domains. In some embodiments, the linker comprises charged amino acids. In some embodiments, the linker is flexible. In some embodiments, the linker comprises at least one glycine and at least one serine. In some embodiments, the linker comprises an amino acid sequence consisting of (Gly₄Ser)₃ (SEQ ID NO: 3). In some embodiments, the linker comprises at least one proline. In some embodiments, the linker comprises an amino acid sequence consisting of SEQ ID NO: 4.

### 3. Polynucleotides

Further provided are polynucleotides encoding the fusion proteins detailed herein. A vector may include the polynucleotide encoding the fusion proteins detailed herein. To obtain expression of a polypeptide, one typically subclones the polynucleotide encoding the polypeptide into an expression vector that contains a promoter to direct transcription, a transcription/translation terminator, and if for a nucleic acid encoding a protein, a ribosome binding site for translational initiation. An example of a vector is pet24 (SEQ ID NO: 12). Suitable bacterial promoters are well known in the art. Further provided is a host cell transformed or transfected with an expression vector comprising a polynucleotide encoding a fusion protein as detailed herein. Bacterial expression systems for expressing the protein are available in, e.g., E. coli, Bacillus sp., and Salmonella (Paiva et al., Gene 1983, 22, 229-235; Mosbach et al., Nature 1983, 302, 543-545). Kits for such expression systems are commercially available. Eukaryotic expression systems for mammalian cells, yeast, and insect cells are well known in the art and are also commercially available. Retroviral expression systems can be used in the present invention. In some embodiments, the fusion protein comprises a polypeptide comprising an amino acid sequence of any one of SEQ ID NOs: 1-11 and 17-19. In some embodiments, the fusion protein comprises a polypeptide encoded by a polynucleotide sequence of any one of SEQ ID NOs: 13-14.

### 4. Administration

The fusion proteins as detailed above can be formulated in accordance with standard techniques well known to those skilled in the pharmaceutical art. Such compositions comprising a fusion protein can be administered in dosages and by techniques well known to those skilled in the medical arts taking into consideration such factors as the age, sex, weight, and condition of the particular subject, and the route of administration.

The fusion protein can be administered prophylactically or therapeutically. In prophylactic administration, the fusion protein can be administered in an amount sufficient to induce a response. In therapeutic applications, the fusion proteins are administered to a subject in need thereof in an amount sufficient to elicit a therapeutic effect. An amount adequate to accomplish this is defined as “therapeutically effective dose.” Amounts effective for this use will depend on, e.g., the particular composition of the fusion protein regimen administered, the manner of administration, the stage and severity of the disease, the general state of health of the patient, and the judgment of the prescribing physician.

The fusion protein can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The fusion protein can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The fusion proteins can be delivered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the fusion protein is administered intravenously, intraarterially, or intraperitoneally to the subject.

The fusion protein can be a liquid preparation such as a suspension, syrup, or elixir. The fusion protein can be incorporated into liposomes, microspheres, or other polymer matrices (such as by a method described in Feigner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The fusion protein may be used as a vaccine. The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation can be carried out via a minimally invasive device.

In some embodiments, the fusion protein is administered in a controlled release formulation. In some embodiments, the fusion protein comprises one or more thermally responsive polypeptides, the thermally responsive polypeptide having a transition temperature such that the fusion protein remains soluble prior to administration and such that the fusion protein transitions upon administration to a gel-like depot in the subject. In some embodiments, the fusion protein comprises one or more thermally responsive polypeptides, the thermally responsive polypeptide having a transition temperature such that the fusion protein remains soluble at room temperature and such that the fusion protein transitions upon administration to a gel-like depot in the subject. For example, in some embodiments, the fusion protein comprises one or more thermally responsive polypeptides, the thermally responsive polypeptide having a transition temperature between room temperature (about 25° C.) and body temperature (about 37° C.), whereby the fusion protein can be administered to form a depot. As used herein, “depot” refers to a gel-like composition comprising a fusion protein that releases the fusion protein over time. In some embodiments, the fusion protein can be injected subcutaneously or intratumorally to form a depot (coacervate). The depot may provide controlled (slow) release of the fusion protein. The depot may provide slow release of the fusion protein into the circulation or the tumor, for example. In some embodiments, the fusion protein may be released from the depot over a period of at least about 1 day, at least about 2 days, at least about 3 days, at least about 4 days, at least about 5 days, at least about 6 days, at least about 7 days, at least about 1 week, at least about 1.5 weeks, at least about 2 weeks, at least about 2.5 weeks, at least about 3.5 weeks, at least about 4 weeks, or at least about 1 month.

### 5. Detection

As used herein, the term “detect” or “determine the presence of” refers to the qualitative measurement of undetectable, low, normal, or high concentrations of one or more fusion proteins, targets, or fusion proteins bound to target. Detection may include in vitro, ex vivo, or in vivo detection. Detection may include detecting the presence of one or more fusion proteins or targets versus the absence of the one or more fusion proteins or targets. Detection may also include quantification of the level of one or more fusion proteins or targets. The term “quantify” or “quantification” may be used interchangeably, and may refer to a process of determining the quantity or abundance of a substance (e.g., fusion protein or target), whether relative or absolute. Any suitable method of detection falls within the general scope of the present disclosure. In some embodiments, the fusion protein comprises a reporter attached thereto for detection. In some embodiments, the fusion protein is labeled with a reporter. In some embodiments, detection of fusion protein bound to target may be determined by methods including but not limited to, band intensity on a Western blot, flow cytometry, radiolabel imaging, cell binding assays, activity assays, SPR, immunoassay, or by various other methods known in the art.

In some embodiments, including those wherein the fusion protein is an antibody mimic for binding and/or detecting a target, any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the fusion protein to the target can be detected via direct labels, attached to the fusion protein or via indirect labels, such as alkaline phosphatase or horseradish peroxidase. The use of immobilized fusion proteins may be incorporated into the immunoassay. The fusion proteins may be immobilized onto a variety of supports, such as magnetic or chromatographic matrix particles, the surface of an assay plate (such as microtiter wells), pieces of a solid substrate material, and the like. An assay strip can be prepared by coating the fusion protein or plurality of fusion proteins in an array on a solid support. This strip can then be dipped into the test biological sample and then processed quickly through washes and detection steps to generate a measurable signal, such as a colored spot.

### 6. Methods

a. Methods of Treating a Disease

The present invention is directed to a method of treating a disease in a subject in need thereof. The method may comprise administering to the subject an effective amount of the fusion protein as described herein. The disease may be selected from cancer, metabolic disease, autoimmune disease, cardiovascular disease, and orthopedic disorders. In some embodiments, the disease is a disease associated with a target of the at least one binding polypeptide.

Metabolic disease may occur when abnormal chemical reactions in the body alter the normal metabolic process. Metabolic diseases may include, for example, insulin resistance, non-alcoholic fatty liver diseases, type 2 diabetes, insulin resistance diseases, cardiovascular diseases, arteriosclerosis, lipid-related metabolic disorders, hyperglycemia, hyperinsulinemia, hyperlipidemia, and glucose metabolic disorders.

Autoimmune diseases arise from an abnormal immune response of the body against substances and tissues normally present in the body. Autoimmune diseases may include, but are not limited to, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, polyarteritis nodosa, myocarditis, psoriasis, Celiac disease, Crohn's disease, ulcerative colitis, and fibromyalgia.

Cardiovascular disease is a class of diseases that involve the heart or blood vessels. Cardiovascular diseases may include, for example, coronary artery diseases (CAD) such as angina and myocardial infarction (heart attack), stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

Orthopedic disorders or musculoskeletal disorders are injuries or pain in the body's joints, ligaments, muscles, nerves, tendons, and structures that support limbs, neck, and back. Orthopedic disorders may include degenerative diseases and inflammatory conditions that cause pain and impair normal activities. Orthopedic disorders may include, for example, carpal tunnel syndrome, epicondylitis, and tendinitis.

Cancers may include, but are not limited to, breast cancer, colorectal cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. In some embodiments, the cancer is colorectal cancer. In some embodiments, the cancer is colorectal adenocarcinoma.

One application of protein therapeutics is cancer treatment. In specific embodiments, the present invention provides a method for using scaffold proteins in developing antibody mimetics for oncological targets of interest. With the emergence of scaffold protein engineering come the possibilities for designing potent protein drugs that are unhindered by steric and architectural limitations. Although potent protein drugs can be invaluable for diagnostics or treatments, successful delivery to the target region can pose a great challenge.

TNF-related apoptosis-inducing ligand receptor 2 (TRAILR-2, also called R5) activates the extrinsic death pathway in a range of human cancer cells (Walczak, et al. Cold Spring Harb. Perspect. Biol., 2013, 5, a008698). TRAILR-2 may be targeted using its natural ligand, TNF-related apoptosis-inducing ligand (TRAIL, also called Apo2L), and other agonists. TRAIL is a homotrimer. TRAIL and other TRAILR-2 agonists may trigger programmed cell death (apoptosis). TRAIL and other TRAILR-2 agonists may have significant anti-tumor activity. However, TRAIL and other TRAILR-2 agonists have not been developed as a clinically efficacious treatment. A possible shortcoming of current TRAIL and other TRAILR-2 agonist therapies may be related to their limited valency. Upon binding of TRAILR-2 to homotrimeric TRAIL, the TRAILR-2 receptor trimerizes and subsequently initiates apoptotic cell death. However, current anti-TRAILR-2 mAbs are only bivalent. Indeed, higher order antibody crosslinking may be required for effective receptor engagement, clustering, and a robust anti-tumor response. Fusion proteins, as detailed herein, that bind multiple TRAILR-2 receptors may provide multivalent agonists capable of forming higher order complexes to treat cancer. FnIII domain has been engineered to have high affinity binding to TRAILR-2. Fusion proteins, as detailed herein, comprising FnIII domains flexible peptide linkers may be used as pro-apoptotic anti-cancer therapeutics. The increased molecular weight and controlled release of the fusion proteins, relative to a binding polypeptide alone, along with the unperturbed potency of the binding polypeptide, may provide a clinically viable option for patients with tumors expressing functional target protein (e.g. TRAILR-2).

In other aspects, provided are methods for treating a disease associated with TNF-related apoptosis-inducing ligand receptor 2 (TRAILR-2) in a subject in need thereof. The method may include administering to the subject an effective amount of a fusion protein as described herein.

b. Methods of Diagnosing a Disease

Provided herein are methods of diagnosing a disease. The methods may include administering to the subject a fusion protein as described herein, and detecting binding of the fusion protein to a target to determine presence of the target in the subject. The presence of the target may indicate the disease in the subject. In other embodiments, the methods may include contacting a sample from the subject with a fusion protein as described herein, determining the level of a target in the sample, and comparing the level of the target in the sample to a control level of the target, wherein a level of the target different from the control level indicates disease in the subject. In some embodiments, the disease is selected from cancer, metabolic disease, autoimmune disease, cardiovascular disease, and orthopedic disorders, as detailed above. In some embodiments, the target comprises a disease marker or biomarker. In some embodiments, the fusion protein may act as an antibody mimic for binding and/or detecting a target.

c. Methods of Determining the Presences of a Target

Provided herein are methods of determining the presence of a target in a sample. The methods may include contacting the sample with a fusion protein as described herein under conditions to allow a complex to form between the fusion protein and the target in the sample, and detecting the presence of the complex. Presence of the complex may be indicative of the target in the sample. In some embodiments, the fusion protein is labeled with a reporter for detection.

In some embodiments, the sample is obtained from a subject and the method further includes diagnosing, prognosticating, or assessing the efficacy of a treatment of the subject. When the method includes assessing the efficacy of a treatment of the subject, then the method may further include modifying the treatment of the subject as needed to improve efficacy.

d. Methods of Determining the Effectiveness of a Treatment

Provided herein are methods of determining the effectiveness of a treatment for a disease in a subject in need thereof. The methods may include contacting a sample from the subject with a fusion protein as detailed herein under conditions to allow a complex to form between the fusion protein and a target in the sample, determining the level of the complex in the sample, wherein the level of the complex is indicative of the level of the target in the sample, and comparing the level of the target in the sample to a control level of the target, wherein if the level of the target is different from the control level, then the treatment is determined to be effective or ineffective in treating the disease.

Time points may include prior to onset of disease, prior to administration of a therapy, various time points during administration of a therapy, and after a therapy has concluded, or a combination thereof. Upon administration of the fusion protein to the subject, the fusion protein may bind a target, wherein the presence of the target indicates the presence of the disease in the subject at the various time points. In some embodiments, the target comprises a disease marker or biomarker. In some embodiments, the fusion protein may act as an antibody mimic for binding and/or detecting a target. Comparison of the binding of the fusion protein to the target at various time points may indicate whether the disease has progressed, whether the diseased has advanced, whether a therapy is working to treat or prevent the disease, or a combination thereof.

In some embodiments, the control level corresponds to the level in the subject at a time point before or during the period when the subject has begun treatment, and the sample is taken from the subject at a later time point. In some embodiments, the sample is taken from the subject at a time point during the period when the subject is undergoing treatment, and the control level corresponds to a disease-free level or to the level at a time point before the period when the subject has begun treatment. In some embodiments, the method further includes modifying the treatment or administering a different treatment to the subject when the treatment is determined to be ineffective in treating the disease.

### 7. Examples

### Example 1

### Design of Multivalent Protein-ELP Fusions

The fusion proteins included two parts (FIG. 1): (i) a multivalent targeting component (e.g., TRAILR-2 agonist or EGFR antagonist) protein in which one or more scaffold protein units (e.g., SEQ ID NO: 1 and 2 or 5) are linked by glycine-serine flexible (e.g., SEQ ID NO: 3) or structured proline-containing linkers (e.g., SEQ ID NO: 4); and (ii) an elastin-like-polypeptide connected to the multivalent protein (e.g., SEQ ID NO: 7-9).

The fusion of (i) to (ii) was at the N- or C-terminus or (ii) was interspersed among (i).

### Example 2

### Design and Preparation of Multivalent Protein-ELP Expression Constructs

The DNA encoding the TRAILR-2-specific Tn3 unit (SEQ ID NO: 13; Swers et al., Mol. Cancer Ther., 2013, 12, 1235-1244) and the EGFR-specific domain (SEQ ID NO: 14; Friedman, et al., J. Mol. Biol. 2008, 376, 1388-1402) were purchased as double-stranded DNA “G-blocks” from Integrated DNA Technologies (Coralville, Iowa). The Tn3 G-block (SEQ ID NO: 13) was amplified using primers “Tn3For” and “Tn3Rev” primers (SEQ ID NO: 15 and 16, respectively). The gene was purchased with a (Gly₄Ser)₃ linker (SEQ ID NO: 3) at the C-terminus and designed with restriction sites compatible with recursive directional (RDL) ligation for seamless cloning of oligomeric genes. The EGFR-binding G-block (SEQ ID NO: 5) was purchased such that it could be inserted into the vector (SEQ ID NO: 12) using Gibson Assembly. The G-block contained 40-50 nucleic acid bases identical to those in the vector.

Enzymes used were from New England Biolabs (Ipswich, Mass.) The amplified Tn3 domain PCR product was purified using the Qiagen (Germantown, Md.) PCR cleanup kit and digested with BseRI for insertion into a BseRI/CIP digested pET-24(+) vector modified for RDL (McDaniel et al. Biomacromolecules, 2010, 11, 944-952). The insert and vector were agarose gel-purified and ligated with QuickLigase to clone the single unit construct. This was followed by digestion of the single unit construct (Tn3 in pET24(+)) with BseRI/CIP and ligation with BseRI-digested insert (Tn3 unit) to clone 2, 4, and 6 Tn3 repeats (written as (Tn3)₂, (Tn3)₄, (Tn3)₆) in the pET-24(+) vector. For cloning the FnIII domain, the G-block was inserted into the BseRI digested/CIP treated pET-24(+) RDL vector using the Gibson Assembly Master Mix (New England Biolabs; Ipswich, Mass.). Subcloning efficiency EB5α cells from EdgeBio (Gaithersburg, Md.) were used for cloning steps.

Once the multivalent Tn3 genes were obtained, the gene for ELP was recombinantly fused to the (Tn3)₆ using RDL. The RDL ligation method for this particular vector called for digestion of the oligomerized Tn3 in modified pET24(+) (SEQ ID NO: 12) with AcuI and BglI, and digestion of ELP (SEQ ID NO: 7-9) in pET24(+) with BseRI and BglI. The digested fragments of DNA were separated using agarose gel electrophoresis, and the DNA bands at the appropriate molecular weights were excised and gel-purified. The resulting fragments were ligated using QuickLigase and successful clones were obtained. The restriction digest scheme mentioned refers to fusion of ELP to the C-terminus of the multivalent agonist, but in some embodiments, the scheme was flipped if N-terminal fusion was desired. In other embodiments, ELP(s) were interspersed between Tn3 repeats with this cloning method. In still other embodiments, an eight-repeat histidine tag (SEQ ID NO: 6) was recombinantly included at the C-terminus for purification and/or analysis purposes. All gene sequences were verified by direct DNA sequencing (Eton Bioscience Inc., Durham, N.C.) prior to expression.

### Example 3

### Expression and Purification of Multivalent TRAILR-2 Agonist-ELP Fusion Proteins

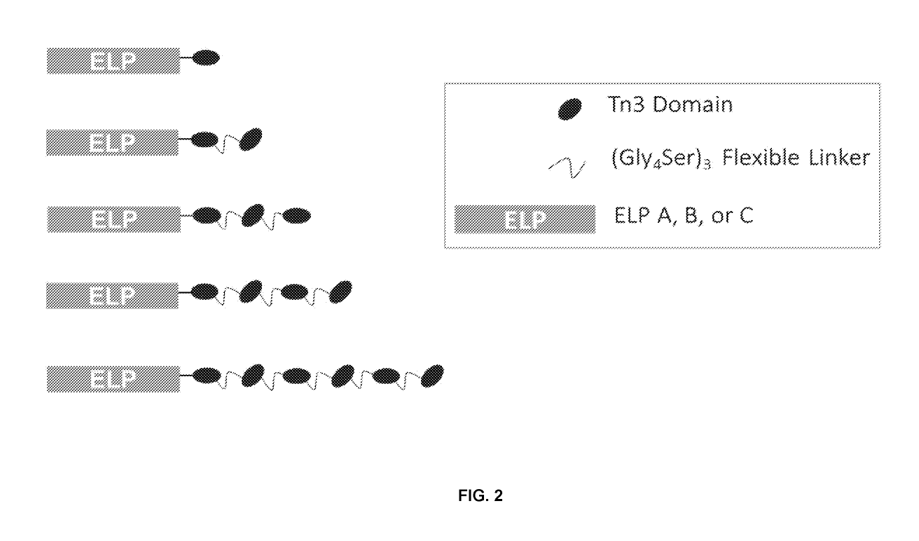

The multivalent ELP-(Tn3)₆ fusion constructs (SEQ ID NO: 10 and 11; FIG. 2) were transformed into BL21(DE3) cells (EMD/Novagen, Gibbstown, N.J.) for expression. Transformants were grown in Terrific Broth (TB) containing 45 μg/mL kanamycin and incubated overnight at 37° C. with shaking. Overnight cultures were diluted 1 to 40 into TB containing 45 μg/mL kanamycin and incubated at 37° C. with shaking for 5-8 hours. Protein expression was then induced by addition of IPTG to 1 mM, and incubation was resumed at 37° C. with shaking. In a specific embodiment, the Tn3-ELP fusion proteins were purified from the cell lysate using inverse transition cycling (ITC) as previously described (Christensen et al., Protein Science 2009, 18, 1377-1387; Hassouneh et al., Methods Enzymol. 2012, 502, 215-37). In another embodiments, C-terminally His₈-tagged ELP-Tn3 fusion proteins were purified from the periplasmic extract using immobilized metal affinity chromatography (IMAC; e.g., HisPur Ni-NTA resin from ThermoFisher Scientific, Pierce, Rockford, Ill.).

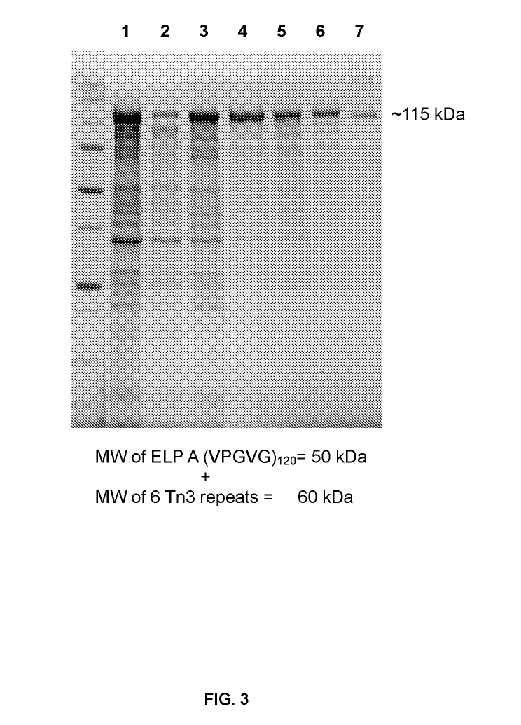

All purified proteins were analyzed by SDS-PAGE on Biorad Mini-PROTEAN TGX Tris-HCl Stain-Free (FIG. 3) or Biorad 4-20% ReadyGel Tris-HCl protein gels for correct molecular weight bands. The protein bands on the latter gel type were visualized with EZBlue Coomassie Brilliant Blue G-250 colloidal protein stain (Sigma Aldrich). Endotoxin was removed from purified protein using an Acrodisc unit with a Mustang E membrane (Pall Corporation, Port Washington, N.Y.).

### Example 4

### In Vitro Testing of Fusion Protein Activity

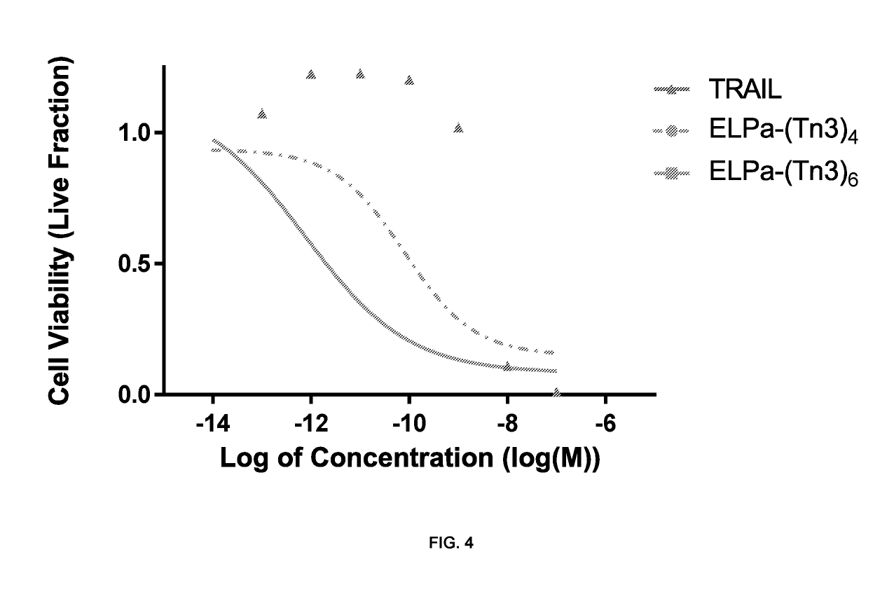

To demonstrate that the multivalent ELP-(Tn3)₆ fusion proteins (SEQ ID NO: 10 and 11) could kill cancer cell lines with the same potency as the non-ELP agonists, the fusions were tested on Colo205 colorectal adenocarcinoma cells. A cell viability assay was performed to calculate an EC₅₀ for the various multivalent fusion proteins (FIG. 4). The EC₅₀ values were comparable to those reported by others for the multivalent agonists.

The cell viability assay was carried out as follows. The Colo205 cells were plated in 96 well plates at a density of 10,000 cells/well in 90 μL of complete media (RPMI 1640+10% FBS+5% HEPES+5% Sodium Pyruvate+P/S) and incubated for 5-4 hours at 37° C. with 5% CO₂. The cells were then treated with 10 μL 20 mM Tris 300 mM L-arginine pH 7 containing a serial dilution of a specific multivalent Tn3-ELP fusion protein or the vehicle control. The treatments were done in triplicate to account for technical variability. After 24-48 hours, the Promega CellTiter 96 Aqueous One Solution Reagent G3581 kit was used according to manufacturer's instructions to assay the number of viable cells using a colorimetric formazan assay method. The inhibition of cell viability was determined using measurements of the absorbance at 490, which is the maximum absorbance wavelength of the formazan product. The dose response curves were generated by plotting inhibition versus compound concentration. The dose response curve was approximated from the scatter plot using a four-parameter logistic model calculation in GraphPad Prism (La Jolla, Calif.), and EC₅₀ was calculated as the concentration of Tn3-ELP required to kill 50% of the Colo205 cells. Fusion of ELPs to the multivalent TRAILR-2 specific Tn3 did not impact their potency (TABLE 1).

TABLE 1
EC₅₀ values for various fusion proteins.

| Fusion Protein | EC₅₀ | |
|---|---|---|
| TRAIL | 2700 | pM |
| (Tn3)₄ | 40 | pM |
| ELPa-(Tn3)₄ | 80 | pM |
| (Tn3)₆ | 1.6 | pM |
| ELPa-(Tn3)₆ | 0.78 | pM |

### Example 5

### Spectrophotometry for Analysis of Fusion Protein Inverse Transition Temperature (Tₜ)

To evaluate the Tt of the fusion proteins, the optical density of the protein solution was monitored at 350 nm (OD350) as a function of temperature. The solution (10-100 μM in 20 mM Tris 300 mM L-arginine, pH 7) was heated at a rate of 1° C./minute using the Cary 300 UV-visible spectrophotometer equipped with a multicell thermoelectric temperature controller (Varian Instruments, Walnut Creek, Calif.). A sharp transition was indicated by the sudden increase in absorbance, and the inflection point of the absorbance versus temperature curve was used to calculate the Tt.

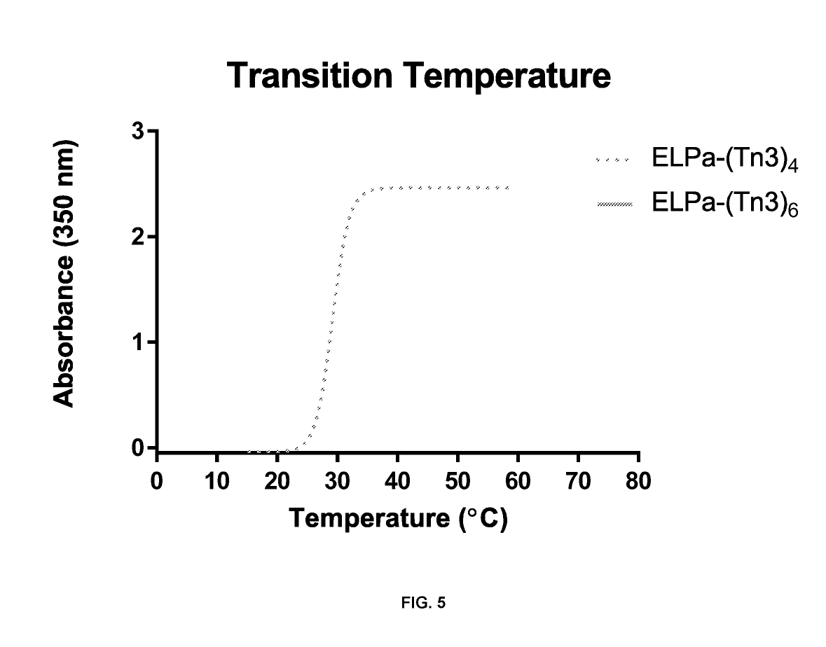

The derivative of the absorbance at 350 nm was calculated with respect to temperature, and the Tt (temperature at maximal turbidity gradient) was obtained. An example set of curves is provided in FIG. 5. The most potent fusions were the 6-repeat Tn3 domain-ELP (SEQ ID NO: 10 and 11, respectively) were chosen for testing in vivo. The hydrophilic ELPb (SEQ ID NO: 8) had a Tt much higher than body temperature; this biopolymer was chosen for fusion to the bioactive protein as a size control. The hydrophobic ELPa (SEQ ID NO: 7) transitioned at 28° C. (see FIG. 5) and formed a gel-like depot upon injection into the mouse.

### Example 6

### Determination of Therapeutic Efficacy In Vivo

Having successfully produced multivalent TRAILR-2 specific ELP-(Tn3)6 fusions that transition to form gel-like depots between room temperature and body temperature, we tested their therapeutic efficacy in a Colo205 colorectal adenocarcinoma mouse xenograft model. One million Colo205 cells (expressing TRAILR-2) were injected subcutaneously into the right flanks of five cohorts of female athymic nude mice. After two weeks, tumors had grown to a volume of approximately 150 mm³, at which a point a single intratumoral injection of 20 mM Tris 300 mM L-arginine pH 7 (vehicle), TRAIL (not shown), depot-forming ELPa-(Tn3)₆ fusion, soluble ELPb-(Tn3)₆ fusion, or soluble (Tn3)₆ was administered. Throughout the experiment, mice were monitored for overall health and activity in accordance with the Duke University Institutional Animal Care & Use Committee. The mice in all treatment groups were dosed at 3.7 μg/mm³ of protein drug and tumor volume was monitored with a digital caliper using the formula:

Volume=0.5×Length×(Width)²

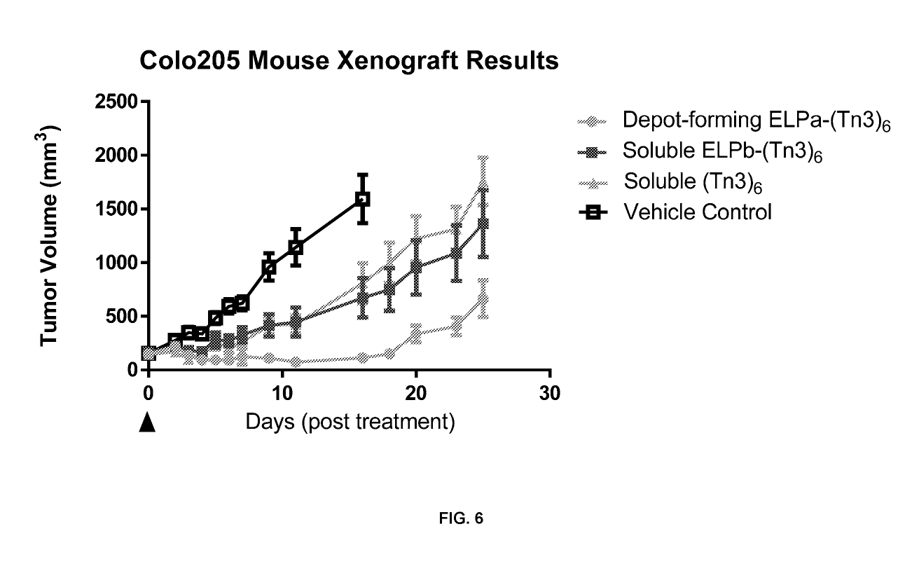

As shown in FIG. 6, the depot-forming ELPa-(Tn3)₆ fusion led to partial tumor regression and slower tumor growth when compared to all other groups. There is a therapeutic advantage of using the depot to release the protein-biopolymer fusion slowly over a longer period of time. This depot approach may be extended to improve the drug delivery of protein-drug conjugates. Also, additional combinations of bioactive multispecific protein-biopolymer fusions can be developed using the methods described herein. The protein architecture, flexibility of the design, and potent therapeutic efficacy make these modular fusions a potential platform for protein delivery.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A fusion protein comprising at least one binding polypeptide and at least one unstructured polypeptide.

Clause 2. The fusion protein of clause 1, wherein the fusion protein comprises a plurality of unstructured polypeptides.

Clause 3. The fusion protein of any one of the preceding clauses, wherein the fusion protein comprises a plurality of binding polypeptides.

Clause 4. The fusion protein of clause 3, further comprising a linker positioned between at least two adjacent binding polypeptides.

Clause 5. The fusion protein of clause 2, further comprising a linker positioned between at least two adjacent unstructured polypeptides.

Clause 6. The fusion protein of any one of clauses 4-5, wherein the linker comprises at least one glycine and at least one serine.

Clause 7. The fusion protein of clause 6, wherein the linker comprises an amino acid sequence consisting of SEQ ID NO: 3 ((Gly₄Ser)₃).

Clause 8. The fusion protein of any one of clauses 4-5, wherein the linker comprises an amino acid sequence consisting of SEQ ID NO: 4.

Clause 9. The fusion protein of any one of clauses 3-8, wherein the plurality of binding polypeptides forms an oligomer.

Clause 10. The fusion protein of any one of clauses 3-9, wherein the binding polypeptide binds a target, and wherein the fusion protein binds more than one target.

Clause 11. The fusion protein of any one of the preceding clauses, wherein the at least one binding polypeptide comprises a Fibronectin type III (FnIII) domain.

Clause 12. The fusion protein of clause 11, wherein the FnIII domain binds TNF-related apoptosis-inducing ligand receptor 2 (TRAILR-2).

Clause 13. The fusion protein of any one of the preceding clauses, wherein the at least one binding polypeptide comprises at least one amino acid sequence of consisting of SEQ ID NO: 17 (RGDS).

Clause 14. The fusion protein of clause 13, wherein the at least one binding polypeptide comprises a plurality of amino acid sequences consisting of SEQ ID NO: 17 (RGDS).

Clause 15. The fusion protein of any one of the preceding clauses, wherein the at least one unstructured polypeptide comprises at least one PG motif comprising an amino acid sequence selected from PG, P(X)ₙG (SEQ ID NO: 18), and (U)ₘP(X)ₙG(Z)ₚ (SEQ ID NO: 20), or a combination thereof, wherein m, n, and p are independently an integer from 1 to 15, and wherein U, X, and Z are independently any amino acid.

Clause 16. The fusion protein of any one of the preceding clauses, wherein the at least one unstructured polypeptide comprises a thermally responsive polypeptide.

Clause 17. The fusion protein of clause 16, wherein the thermally responsive polypeptide comprises an elastin-like polypeptide (ELP).

Clause 18. The fusion protein of any one of the preceding clauses, wherein the at least one unstructured polypeptide comprises an amino acid sequence consisting of (VPGXG)ₙ (SEQ ID NO: 19), wherein X is any amino acid except proline and n is an integer greater than or equal to 1.

Clause 19. The fusion protein of clause 18, wherein n is 60, 120, or 180.

Clause 20. The fusion protein of clause 18, wherein X is valine.

Clause 21. The fusion protein of any one of the preceding clauses, further comprising at least one linker positioned between the at least one binding polypeptide and the at least one unstructured polypeptide.

Clause 22. The fusion protein of clause 21, wherein the fusion protein comprises a plurality of linkers between the at least one binding polypeptide and the at least one unstructured polypeptide.

Clause 23. The fusion protein of any one of the preceding clauses, wherein the at least one binding polypeptide is positioned N-terminal to the at least one unstructured polypeptide.

Clause 24. The fusion protein of any one of clauses 1-23 wherein the at least one binding polypeptide is positioned C-terminal to the at least one unstructured polypeptide.

Clause 25. The fusion protein of any one of the preceding clauses, wherein the at least one unstructured polypeptide has a LCST between about 0° C. and about 100° C.

Clause 26. The fusion protein of any one of the preceding clauses, wherein the at least one unstructured polypeptide has a UCST between about 0° C. and about 100° C.

Clause 27. A method for treating a disease in a subject in need thereof, the method comprising administering to the subject an effective amount of the fusion protein according to any one of the preceding clauses.

Clause 28. The method of clause 27, wherein the fusion protein is administered in a controlled release formulation.

Clause 29. The method of clause 27, wherein the fusion protein forms a depot upon administration to the subject.

Clause 30. The method of any one of clauses 27-28, wherein the fusion protein is administered intravenously, intraarterially, or intraperitoneally to the subject.

Clause 31. The method of any one of clauses 27-30, wherein the disease comprises cancer.

Clause 32. The method of clause 31, wherein the fusion protein is administered intratumorally.

Clause 33. The method of any one of clauses 27-32, wherein the cancer is colorectal adenocarcinoma.

Clause 34. The method of any one of clauses 27-33, wherein the at least one binding polypeptide comprises an FnIII domain or a plurality of FnIII domains, and wherein the disease is a disease associated with TRAILR-2.

Clause 35. The method of any one of clauses 27-34, wherein the disease is a disease associated with a target of the at least one binding polypeptide.

Clause 36. A multivalent fusion protein comprising at least one Fibronectin type III (FnIII) domain and at least one elastin-like polypeptide (ELP), wherein the FnIII domain binds TNF-related apoptosis-inducing ligand receptor 2 (TRAILR-2).

Clause 37. The multivalent fusion protein of clause 36, wherein the at least one ELP comprises an amino acid sequence consisting of (VPGXG)ₙ (SEQ ID NO: 19), wherein X is any amino acid except proline and n is an integer greater than or equal to 1.

Clause 38. The multivalent fusion protein of clause 37, wherein n is 60, 120, or 180.

Clause 39. The multivalent fusion protein of clause 37, wherein X is valine.

Clause 40. The multivalent fusion protein of any one of clauses 36-39, wherein the at least one FnIII domain comprises an amino acid sequence consisting of SEQ ID NO: 1.

Clause 41. The multivalent fusion protein of any one of clauses 36-40, wherein the multivalent fusion protein comprises a plurality of FnIII domains.

Clause 42. The multivalent fusion protein of clause 41, wherein the multivalent fusion protein comprises 2, 4, or 6 FnIII domains.

Clause 43. The multivalent fusion protein of clause 41 or 42, wherein the multivalent fusion protein further comprises a linker positioned between at least two adjacent FnIII domains.

Clause 44. The multivalent fusion protein of clause 43, wherein the linker comprises at least one glycine and at least one serine.

Clause 45. The multivalent fusion protein of clause 44, wherein the linker comprises an amino acid sequence consisting of SEQ ID NO: 3 ((Gly₄Ser)₃).

Clause 46. The multivalent fusion protein of clause 43, wherein the linker comprises an amino acid sequence consisting of SEQ ID NO: 4.

Clause 47. A method for treating a disease associated with TNF-related apoptosis-inducing ligand receptor 2 (TRAILR-2) in a subject in need thereof, the method comprising administering to the subject an effective amount of the multivalent fusion protein of any one of clauses 36-46.

Clause 48. The method of clause 47, wherein the disease comprises cancer.

Clause 49. The method of clause 48, wherein the cancer comprises colorectal adenocarcinoma.

Clause 50. The method of any one of clauses 47-49, wherein the multivalent fusion protein is administered intravenously, intraarterially, or intraperitoneally to the subject.

Clause 51. The method of any one of clauses 48-49, wherein the multivalent fusion protein is administered intratumorally.

Clause 52. The method of any one of clauses 47-51, wherein the multivalent fusion protein forms a depot upon administration to the subject.

Clause 53. The method of any one of clauses 47-51, wherein the multivalent fusion protein is administered in a controlled release formulation.

Clause 54. A method of diagnosing a disease in a subject, the method comprising contacting a sample from the subject with the fusion protein according to any one of clauses 1-26; and detecting binding of the fusion protein to a target to determine presence of the target in the sample, wherein the presence of the target in the sample indicates the disease in the subject.

Clause 55. The method of clause 54, wherein the disease is selected from cancer, metabolic disease, autoimmune disease, cardiovascular disease, and orthopedic disorder.

Clause 56. A method of determining the presence of a target in a sample, the method comprising contacting the sample with the fusion protein of any one of clauses 1-26 under conditions to allow a complex to form between the fusion protein and the target in the sample; and detecting the presence of the complex, wherein presence of the complex is indicative of the target in the sample.

Clause 57. The method of clause 56, wherein the sample is obtained from a subject and the method further comprises diagnosing a disease, prognosticating, or assessing the efficacy of a treatment of the subject.

Clause 58. The method of clause 57, wherein when the method further comprises assessing the efficacy of a treatment of the subject, then the method further comprises modifying the treatment of the subject as needed to improve efficacy.

Clause 59. A method of determining the effectiveness of a treatment for a disease in a subject in need thereof, the method comprising contacting a sample from the subject with the fusion protein of any one of clauses 1-26 under conditions to allow a complex to form between the fusion protein and a target in the sample; determining the level of the complex in the sample, wherein the level of the complex is indicative of the level of the target in the sample; and comparing the level of the target in the sample to a control level of the target, wherein if the level of the target is different from the control level, then the treatment is determined to be effective or ineffective in treating the disease.

Clause 60. A method of diagnosing a disease in a subject, the method comprising: contacting a sample from the subject with the fusion protein of any one of clauses 1-26; determining the level of a target in the sample; and comparing the level of the target in the sample to a control level of the target, wherein a level of the target different from the control level indicates disease in the subject.

Clause 61. The method of clause 59 or 60, wherein the control level corresponds to the level in the subject at a time point before or during the period when the subject has begun treatment, and wherein the sample is taken from the subject at a later time point.

Clause 62. The method of clause 59 or 60, wherein the sample is taken from the subject at a time point during the period when the subject is undergoing treatment, and wherein the control level corresponds to a disease-free level or to the level at a time point before the period when the subject has begun treatment.

Clause 63. The method of any one of clauses 59 and 61-62, the method further comprising modifying the treatment or administering a different treatment to the subject when the treatment is determined to be ineffective in treating the disease.

Clause 64. The method of any one of clauses 54-63, wherein the fusion protein is labeled with a reporter.

Clause 65. The method of any one of clauses 54-64, wherein the disease is selected from cancer, metabolic disease, autoimmune disease, cardiovascular disease, and orthopedic disorder.

SEQUENCES

SEQ ID NO: 1
TRAILR2-Specific Tn3, polypeptide
GAIEVKDVTDTTALITWAKPWVDPPPLWGCELTYGIKDVPGDRTTIDLQQKHTAYSIGNLKPDT
EYEVSLICFDPYGMRSKPAKETFTT
SEQ ID NO: 2
TRAILR2-Specific Tn3 Sequence without Cysteines, polypeptide
GAIEVKDVTDTTALITWAKPWVDPPPLWGIELTYGIKDVPGDRTTIDLQQKHTAYSIGNLKPDTE
YEVSLISFDPYGMRSKPAKETFTT
SEQ ID NO: 3
Flexible GlySer Linker, polypeptide
GGGGSGGGGSGGGGS
SEQ ID NO: 4
Proline-Containing Linker, polypeptide
PQPQPKPQPKPEPEPQPQG
SEQ ID NO: 5
EGFR-Binding Domain, polypeptide
GVDNKFNKEMWAAWEEIRNLPNLNGWQMTAFIASLVDDPSQSANLLAEAKKLNDAQAPKG
SEQ ID NO: 6
His-8 Tag, polypeptide
HHHHHHHH
SEQ ID NO: 7
ELP A, polypeptide
VGVPGVGVPGVGVPGVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGVPGVGVPGVGVPG
VGVPGVGVPGVGVPGVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGVPGVGVPGVGVPG
VGVPGVGVPG
SEQ ID NO: 8
ELP B, polypeptide
VPGGGVPGAGVPGGGVPGAGVPGGGVPGAGVPGGGVPGAGVPGGGVPGAGVPGGGVPG
AGVPGGGVPGAGVPGGGVPGAGVPGGGVPGAGVPGGGVPGAGVPGGGVPGAGVPGGGV
PGAGVPGGGVPGAGVPGGGVPGAGVPGGGVPGAGVPGGGVPGAGVPGGGVPGAGVPGG
GVPGAGVPGGGVPGAGVPGGGVPGAGVPGGGVPGAGVPGGGVPGAGVPGGGVPGAGVP
GGGVPGAGVPGGGVPGAGVPGGGVPGAGVPGGGVPGAGVPGGGVPGAGVPGGGVPGAG
VPGGGVPGAGVPGGGVPGAGVPGGGVPGAGVPGGGVPGAGVPGGGVPGAGVPGGGVPG
AGVPGGGVPGAGVPGGGVPGAGVPGGGVPGAGVPGGGVPGAGVPGGGVPGAGVPGGGV
PGAGVPGGGVPGAGVPGGGVPGAGVPGGGVPGAGVPGGGVPGAGVPGGGVPGAGVPGG
GVPGAGVPGGGVPGAGVPGGGVPGAGVPGGGVPGAGVPGGGVPGAGVPGGGVPGAGVP
GGGVPGAGVPGGGVPGAGVPGGGVPGAGVPGGGVPGAGVPGGGVPGAGVPGGGVPGAG
VPGGGVPGAGVPGGGVPGAG
SEQ ID NO: 9
ELP C, polypeptide
VGVPGVGVPGVGVPGVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGVPGVGVPGVGVPG
VGVPG
SEQ ID NO: 10
ELPa-(Tn3)₆, polypeptide
VGVPGVGVPGVGVPGVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGVPGVGVPGVGVPG
VGVPGVGVPGVGVPGVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGVPGVGVPGVGVPG
VGVPGVGVPGAIEVKDVTDTTALITWAKPWVDPPPLWGCELTYGIKDVPGDRTTIDLQQKHTA
YSIGNLKPDTEYEVSLICFDPYGMRSKPAKETFTTGGGGSGGGGSGGGGSGAIEVKDVTDTTA
LITWAKPWVDPPPLWGCELTYGIKDVPGDRTTIDLQQKHTAYSIGNLKPDTEYEVSLICFDPYG
MRSKPAKETFTTGGGGSGGGGSGGGGSGAIEVKDVTDTTALITWAKPWVDPPPLWGCELTY
GIKDVPGDRTTIDLQQKHTAYSIGNLKPDTEYEVSLICFDPYGMRSKPAKETFTTGGGGSGGG
GSGGGGSGAIEVKDVTDTTALITWAKPWVDPPPLWGCELTYGIKDVPGDRTTIDLQQKHTAYSI
GNLKPDTEYEVSLICFDPYGMRSKPAKETFTTGGGGSGGGGSGGGGSGAIEVKDVTDTTALIT
WAKPWVDPPPLWGCELTYGIKDVPGDRTTIDLQQKHTAYSIGNLKPDTEYEVSLICFDPYGMR
SKPAKETFTTGGGGSGGGGSGGGGSGAIEVKDVTDTTALITWAKPWVDPPPLWGCELTYGIK
DVPGDRTTIDLQQKHTAYSIGNLKPDTEYEVSLICFDPYGMRSKPAKETFTTGGGGSGGGGSG
GGGS
SEQ ID NO: 11
ELPb-(Tn3)₆, polypeptide
VPGGGVPGAGVPGGGVPGAGVPGGGVPGAGVPGGGVPGAGVPGGGVPGAGVPGGGVPG
AGVPGGGVPGAGVPGGGVPGAGVPGGGVPGAGVPGGGVPGAGVPGGGVPGAGVPGGGV
PGAGVPGGGVPGAGVPGGGVPGAGVPGGGVPGAGVPGGGVPGAGVPGGGVPGAGVPGG
GVPGAGVPGGGVPGAGVPGGGVPGAGVPGGGVPGAGVPGGGVPGAGVPGGGVPGAGVP
GGGVPGAGVPGGGVPGAGVPGGGVPGAGVPGGGVPGAGVPGGGVPGAGVPGGGVPGAG
VPGGGVPGAGVPGGGVPGAGVPGGGVPGAGVPGGGVPGAGVPGGGVPGAGVPGGGVPG
AGVPGGGVPGAGVPGGGVPGAGVPGGGVPGAGVPGGGVPGAGVPGGGVPGAGVPGGGV
PGAGVPGGGVPGAGVPGGGVPGAGVPGGGVPGAGVPGGGVPGAGVPGGGVPGAGVPGG
GVPGAGVPGGGVPGAGVPGGGVPGAGVPGGGVPGAGVPGGGVPGAGVPGGGVPGAGVP
GGGVPGAGVPGGGVPGAGVPGGGVPGAGVPGGGVPGAGVPGGGVPGAGVPGGGVPGAG
VPGGGVPGAGVPGGGVPGAGAIEVKDVTDTTALITWAKPWVDPPPLWGCELTYGIKDVPGDR
TTIDLQQKHTAYSIGNLKPDTEYEVSLICFDPYGMRSKPAKETFTTGGGGSGGGGSGGGGSGA
IEVKDVTDTTALITWAKPWVDPPPLWGCELTYGIKDVPGDRTTIDLQQKHTAYSIGNLKPDTEYE
VSLICFDPYGMRSKPAKETFTTGGGGSGGGGSGGGGSGAIEVKDVTDTTALITWAKPWVDPP
PLWGCELTYGIKDVPGDRTTIDLQQKHTAYSIGNLKPDTEYEVSLICFDPYGMRSKPAKETFTT
GGGGSGGGGSGGGGSGAIEVKDVTDTTALITWAKPWVDPPPLWGCELTYGIKDVPGDRTTID
LQQKHTAYSIGNLKPDTEYEVSLICFDPYGMRSKPAKETFTTGGGGSGGGGSGGGGSGAIEVK
DVTDTTALITWAKPWVDPPPLWGCELTYGIKDVPGDRTTIDLQQKHTAYSIGNLKPDTEYEVSLI
CFDPYGMRSKPAKETFTTGGGGSGGGGSGGGGSGAIEVKDVTDTTALITWAKPWVDPPPLW
GCELTYGIKDVPGDRTTIDLQQKHTAYSIGNLKPDTEYEVSLICFDPYGMRSKPAKETFTTGGG
GSGGGGSGGGGS
SEQ ID NO: 12
pet24 Vector, polynucleotide
TGGCGAATGGGACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTTTAG
GGTTCCGATTTAGTGCTTTACGGCACCTCGACCCCAAAAAACTTGATTAGGGTGATGGTTC
ACGTAGTGGGCCATCGCCCTGATAGACGGTTTTTCGCCCTTTGACGTTGGAGTCCACGTTC
TTTAATAGTGGACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGATTTAACAAAA
ATTTAACGCGAATTTTAACAAAATATTAACGTTTACAATTTCAGGTGGCACTTTTCGGGGAA
ATGTGCGCGGAACCCCTATTTGTTTATTTTTCTAAATACATTCAAATATGTATCCGCTCATGA
ATTAATTCTTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGGCAGTT
CCATAGGATGGCAAGATCCTGGTATCGGTCTGCGATTCCGACTCGTCCAACATCAATACAA
CCTATTAATTTCCCCTCGTCAAAAATAAGGTTATCAAGTGAGAAATCACCATGAGTGACGAC
TGAATCCGGTGAGAATGGCAAAAGTTTATGCATTTCTTTCCAGACTTGTTCAACAGGCCAG
CCATTACGCTCGTCATCAAAATCACTCGCATCAACCAAACCGTTATTCATTCGTGATTGCGC
CTGAGCGAGACGAAATACGCGATCGCTGTTAAAAGGACAATTACAAACAGGAATCGAATGC
AACCGGCGCAGGAACACTGCCAGCGCATCAACAATATTTTCACCTGAATCAGGATATTCTT
CTAATACCTGGAATGCTGTTTTCCCGGGGATCGCAGTGGTGAGTAACCATGCATCATCAGG
AGTACGGATAAAATGCTTGATGGTCGGAAGAGGCATAAATTCCGTCAGCCAGTTTAGTCTG
ACCATCTCATCTGTAACATCATTGGCAACGCTACCTTTGCCATGTTTCAGAAACAACTCTGG
CGCATCGGGCTTCCCATACAATCGATAGATTGTCGCACCTGATTGCCCGACATTATCGCGA
GCCCATTTATACCCATATAAATCAGCATCCATGTTGGAATTTAATCGCGGCCTAGAGCAAGA
CGTTTCCCGTTGAATATGGCTCATAACACCCCTTGTATTACTGTTTATGTAAGCAGACAGTT
TTATTGTTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTCTAGTGTAGCCG
TAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAGCCT
ATGGAAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCCTTTGAGTG
AGCTGATACCGCTCGCCGCAGCCGAACGACCGAGCGCAGCGAGTCAGTGAGCGAGGAAG
CGGAAGAGCGCCTGATGCGGTATTTTCTCCTTACGCATCTGTGCGGTATTTCACACCGCAT
ATATGGTGCACTCTCAGTACAATCTGCTCTGATGCCGCATAGTTAAGCCAGTATACACTCC
GCTATCGCTACGTGACTGGGTCATGGCTGCGCCCCGACACCCGCCAACACCCGCTGACG
CGCCCTGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGAGGCAGCTGCGGT
AAAGCTCATCAGCGTGGTCGTGAAGCGATTCACAGATGTCTGCCTGTTCATCCGCGTCCA
GCTCGTTGAGTTTCTCCAGAAGCGTTAATGTCTGGCTTCTGATAAAGCGGGCCATGTTAAG
GGCGGTTTTTTCCTGTTTGGTCACTGATGCCTCCGTGTAAGGGGGATTTCTGTTCATGGGG
GTAATGATACCGATGAAACGAGAGAGGATGCTCACGATACGGGTTACTGATGATGAACATG
CCCGGTTACTGGAACGTTGTGAGGGTAAACAACTGGCGGTATGGATGCGGCGGGACCAG
AGAAAAATCACTCAGGGTCAATGCCAGCGCTTCGTTAATACAGATGTAGGTGTTCCACAGG
GTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGC
AGACGTTTTGCAGCAGCAGTCGCTTCACGTTCGCTCGCGTATCGGTGATTCATTCTGCTAA
CCAGTAAGGCAACCCCGCCAGCCTAGCCGGGTCCTCAACGACAGGAGCACGATCATGCG
CACCCGTGGGGCCGCCATGCCGGCGATAATGGCCTGCTTCTCGCCGAAACGTTTGGTGG
CGGGACCAGTGACGAAGGCTTGAGCGAGGGCGTGCAAGATTCCGAATACCGCAAGCGAC
AGGCCGATCATCGTCGCGCTCCAGCGAAAGCGGTCCTCGCCGAAAATGACCCAGAGCGC
TGCCGGCACCTGTCCTACGAGTTGCATGATAAAGAAGACAGTCATAAGTGCGGCGACGAT
AGTCATGCCCCGCGCCCACCGGAAGGAGCTGACTGGGTTGAAGGCTCTCAAGGGCATCG
GTCGAGATCCCGGTGCCTAATGAGTGAGCTAACTTACATTAATTGCGTTGCGCTCACTGCC
CGCTTTCCAGTCGGGAAACCTGTCGTGCCAGCTGCATTAATGAATCGGCCAACGCGCGGG
GAGAGGCGGTTTGCGTATTGGGCGCCAGGGTGGTTTTTCTTTTCACCAGTGAGACGGGCA
ACAGCTGATTGCCCTTCACCGCCTGGCCCTGAGAGAGTTGCAGCAAGCGGTCCACGCTGG
TTTGCCCCAGCAGGCGAAAATCCTGTTTGATGGTGGTTAACGGCGGGATATAACATGAGCT
GTCTTCGGTATCGTCGTATCCCACTACCGAGATATCCGCACCAACGCGCAGCCCGGACTC
GGTAATGGCGCGCATTGCGCCCAGCGCCATCTGATCGTTGGCAACCAGCATCGCAGTGG
GAACGATGCCCTCATTCAGCATTTGCATGGTTTGTTGAAAACCGGACATGGCACTCCAGTC
GCCTTCCCGTTCCGCTATCGGCTGAATTTGATTGCGAGTGAGATATTTATGCCAGCCAGCC
AGACGCAGACGCGCCGAGACAGAACTTAATGGGCCCGCTAACAGCGCGATTTGCTGGTGA
CCCAATGCGACCAGATGCTCCACGCCCAGTCGCGTACCGTCTTCATGGGAGAAAATAATA
CTGTTGATGGGTGTCTGGTCAGAGACATCAAGAAATAACGCCGGAACATTAGTGCAGGCA
GCTTCCACAGCAATGGCATCCTGGTCATCCAGCGGATAGTTAATGATCAGCCCACTGACG
CGTTGCGCGAGAAGATTGTGCACCGCCGCTTTACAGGCTTCGACGCCGCTTCGTTCTACC
ATCGACACCACCACGCTGGCACCCAGTTGATCGGCGCGAGATTTAATCGCCGCGACAATT
TGCGACGGCGCGTGCAGGGCCAGACTGGAGGTGGCAACGCCAATCAGCAACGACTGTTT
GCCCGCCAGTTGTTGTGCCACGCGGTTGGGAATGTAATTCAGCTCCGCCATCGCCGCTTC
CACTTTTTCCCGCGTTTTCGCAGAAACGTGGCTGGCCTGGTTCACCACGCGGGAAACGGT
CTGATAAGAGACACCGGCATACTCTGCGACATCGTATAACGTTACTGGTTTCACATTCACC
ACCCTGAATTGACTCTCTTCCGGGCGCTATCATGCCATACCGCGAAAGGTTTTGCGCCATT
CGATGGTGTCCGGGATCTCGACGCTCTCCCTTATGCGACTCCTGCATTAGGAAGCAGCCC
AGTAGTAGGTTGAGGCCGTTGAGCACCGCCGCCGCAAGGAATGGTGCATGCAAGGAGAT
GGCGCCCAACAGTCCCCCGGCCACGGGGCCTGCCACCATACCCACGCCGAAACAAGCGC
TCATGAGCCCGAAGTGGCGAGCCCGATCTTCCCCATCGGTGATGTCGGCGATATAGGCGC
CAGCAACCGCACCTGTGGCGCCGGTGATGCCGGCCACGATGCGTCCGGCGTAGAGGATC
GAGATCTCGATCCCGCGAAATTAATACGACTCACTATAGGGGAATTGTGAGCGGATAACAA
TTCCCCTCTAGAAATAATTTTGTTTAACTTTAAGAAGGAGGAGTACATATGGGCTGATGATA
ATGATCTTCAGGATCCGAATTCGAGCTCCGTCGACAAGCTTGCGGCCGCACTCGAGCACC
ACCACCACCACCACTGAGATCCGGCTGCTAACAAAGCCCGAAAGGAAGCTGAGTTGGCTG
CTGCCACCGCTGAGCAATAACTAGCATAACCCCTTGGGGCCTCTAAACGGGTCTTGAGGG
GTTTTTTGCTGAAAGGAGGAACTATATCCGGAT
SEQ ID NO: 13
Tn3 G-block, polynucleotide
TAAGAAGGAGGAGTACATATGGGCGCTATCGAAGTTAAAGACGTTACCGACACCACCGCT
CTGATCACCTGGGCTAAACCGTGGGTTGACCCGCCGCCGCTGTGGGGTTGCGAACTGAC
CTACGGTATCAAAGACGTTCCGGGTGACCGTACCACCATCGACCTGCAGCAGAAACACAC
CGCTTACTCTATCGGTAACCTGAAACCGGACACCGAATACGAAGTTTCTCTGATCTGCTTC
GACCCGTACGGTATGCGTTCTAAACCGGCTAAAGAAACCTTCACCACCGGTGGTGGTGGT
TCTGGTGGTGGTGGTTCTGGTGGTGGTGGTTCTGGCATATGTACTCCTCCTTA
SEQ ID NO: 14
EGFR Binding Domain G-block, polynucleotide
AGAAATAATTTTGTTTAACTTTAAGAAGGAGGAGTACATATGGGCGTTGATAACAAATTCAA
TAAAGAAATGTGGGCAGCCTGGGAAGAAATTCGTAACCTGCCGAACCTGAATGGTTGGCA
AATGACCGCCTTCATTGCGAGCCTGGTGGATGATCCGAGCCAAAGCGCTAATCTGCTGGC
GGAAGCGAAAAAACTGAACGACGCCCAAGCCCCGAAAGGCTGATAATAATGATCTTCAGG
ATCCGAATTCGAGCTCCGTC
SEQ ID NO: 15
Tn3 Forward Amplification Primer, polynucleotide
TAAGAAGGAGGAGTACATATGGGCGC
SEQ ID NO: 16
Tn3 Reverse Amplification Primer, polynucleotide
TAAGGAGGAGTACATATGCCAGAACCAC
SEQ ID NO: 17
Linker, polypeptide
RGDS
SEQ ID NO: 18
A PG motif, polypeptide, wherein X is any amino acid and n is an integer from 1 to 15
P(X)ₙG
SEQ ID NO: 19
ELP repeat, polypeptide, wherein X is any amino acid except proline and n is an integer greater
than or equal to 1
(VPGXG)ₙ
SEQ ID NO: 20
A PG motif, polypeptide, wherein U, X, and Z are independently any amino acid and m, n, and p
are independently an integer from 1 to 15
(U)ₘP(X)ₙG(Z)ₚ

We claim:

1-35. (canceled)

36. A multivalent fusion protein comprising at least one Fibronectin type III (FnIII) domain and at least one elastin-like polypeptide (ELP), wherein the FnIII domain binds TNF-related apoptosis-inducing ligand receptor 2 (TRAILR-2), and comprises an amino acid sequence consisting of SEQ ID NO: 1.

37. The multivalent fusion protein of claim 36, wherein the at least one ELP comprises an amino acid sequence consisting of (VPGXG)ₙ (SEQ ID NO: 19), wherein X is any amino acid except proline and n is an integer greater than or equal to 1.

38. The multivalent fusion protein of claim 37, wherein n is 60, 120, or 180.

39. The multivalent fusion protein of claim 37, wherein X is valine.

40. (canceled)

41. The multivalent fusion protein of claim 36, wherein the multivalent fusion protein comprises a plurality of FnIII domains.

42. The multivalent fusion protein of claim 41, wherein the multivalent fusion protein comprises 2, 4, or 6 FnIII domains.

43. The multivalent fusion protein of claim 41, wherein the multivalent fusion protein further comprises a linker positioned between at least two adjacent FnIII domains.

44. The multivalent fusion protein of claim 43, wherein the linker comprises at least one glycine and at least one serine.

45. The multivalent fusion protein of claim 44, wherein the linker comprises an amino acid sequence consisting of SEQ ID NO: 3 ((Gly₄Ser)₃).

46. The multivalent fusion protein of claim 43, wherein the linker comprises an amino acid sequence consisting of SEQ ID NO: 4.

47. A method for treating a disease associated with TNF-related apoptosis-inducing ligand receptor 2 (TRAILR-2) in a subject in need thereof, the method comprising administering to the subject an effective amount of the multivalent fusion protein of claim 36.

48. The method of claim 47, wherein the disease comprises cancer.

49. The method of claim 48, wherein the cancer comprises colorectal adenocarcinoma.

50. The method of claim 47, wherein the multivalent fusion protein is administered intravenously, intraarterially, or intraperitoneally to the subject.

51. The method of claim 48, wherein the multivalent fusion protein is administered intratumorally.

52. The method of claim 47, wherein the multivalent fusion protein forms a depot upon administration to the subject.

53. The method of claim 47, wherein the multivalent fusion protein is administered in a controlled release formulation.

54-65. (canceled)
